(19) United States
(12) Patent Application Publication
(10) Pub. No.: US 2023/0320347 A1
(43) Pub. Date: Oct. 12, 2023

(54) FLOCCULANT BASED DISINFECTION PROCESS FOR PATHOGENIC MEDICAL WASTE DISPOSAL

(71) Applicant: COUNCIL OF SCIENTIFIC AND INDUSTRIAL RESEARCH, New Delhi (IN)

(72) Inventors: Sreejith SHANKAR POOPPANAL, Pappanamcode, Kerala, Thiruvananthapuram (IN); Hareesh UNNIKRISHNAN NAIR SARASWATHY, Pappanamcode, Kerala, Thiruvananthapuram (IN); Binod PARAMESWARAN, Pappanamcode, Kerala, Thiruvananthapuram (IN); Rajeev Kumar SUKUMARAN, Pappanamcode, Kerala, Thiruvananthapuram (IN); Ayyappanpillai AJAYAGHOSH, Pappanamcode, Kerala, Thiruvananthapuram (IN)

(21) Appl. No.: 18/044,269

(22) PCT Filed: Jan. 13, 2021
(86) PCT No.: PCT/IN2021/050032

§ 371 Date: Mar. 7, 2023

(30) Foreign Application Priority Data
Sep. 8, 2020 (IN) 202011039050

(51) Int. Cl. A01N 25/04; A01P 1/00; A61L 2/18; A61L 11/00; A01N 37/46; A01N 59/16; G01N 1/34; A01N 59/00; A01N 59/06; B09B 3/70

(52) U.S. Cl. CPC A01N 25/04; A01P 1/00; A61L 2/18; A61L 11/00; A01N 37/46; A01N 59/16; G01N 1/34; A01N 59/00; A01N 59/06; B09B 3/70; A61L 2202/24; B09B 2101/65

(57) ABSTRACT

Mismanagement of infectious wastes such as test samples leads to the transmission of microbes/toxins/viruses and spread of contagious and infectious diseases. Adding a flocculating agent to liquid waste reduces the risk of spills and aerosolization. Provide is a flocculating/gelating agent comprising the sol of a selected nanomaterial with a defined weight composition in water and a poly-amino acid (polyglutamic acid), that is capable of instantaneous flocculation/gelation, thereby disinfecting both liquid as well as solid samples, rendering them non-infectious, with >99.9% microbial disinfection. Segregation, transportation and incineration of such disinfected medical wastes are easier, safer and decrease medical waste disposal costs for a healthcare facility.

### FIELD OF THE INVENTION

The present invention relates to a flocculant based gelation-solidification-disinfection system for the treatment of biomedical waste. Particularly, the present invention relates to the process for preparation of disinfecting composition comprising of a selected nanomaterial as its sol in water and a poly-amino acid containing a basifying agent, which when mixed with solid or fluid waste samples at a defined volumetric and/or weighted composition leads to instantaneous flocculation/gelation/solidification with >99.9% microbial disinfection. More particularly, the present invention relates to a disinfecting device for the treatment of biomedical waste.

### BACKGROUND AND PRIOR ART OF THE INVENTION

Mismanagement of infectious wastes such as biomedical test samples leads to the transmission of microbes/toxins/viruses and further steer the spread of contagious and infectious diseases. According to a position statement (2000) by WHO, improper management of medical wastes such as infected hypodermic needles and syringes has caused infections pertaining to hepatitis B (21 million cases), hepatitis C (2 million cases) and HIV (0.26 million cases) worldwide. The following statements quoted from WHO undermine the significance and the need for proper medical waste management: “Poor management of medical waste potentially exposes healthcare workers, waste handlers, patients and the community at large to infection, toxic effects and injuries, and risks polluting the environment. It is essential that all medical waste materials are segregated at the point of generation, appropriately treated, and disposed of safely” (reproduced from http://www.who.int/topics/medical_waste/en/).

Adding a flocculating agent to liquid waste reduces the risk of spills and aerosolization. Solid wastes such as cotton, sharps as well as tissue papers may also lead to spread of infections, further, simple absorbers or hypochlorites that are currently in use are not always capable of treating such wastes. If the flocculating/gelling agent contains a disinfectant, it may be possible to dispose of the waste as non-regulated medical waste, which is less expensive than red-bagging. Segregation, transportation and incineration of such disinfected medical wastes are easier, safer and also decreases the medical waste disposal costs for a healthcare facility.

Several strategies have been adopted for the management of liquid biomedical waste and include, but not limited to, sanitary sewer disposal methods, chemical treatments using 1% sodium hypochlorite solution with a minimum contact period of 30 minutes or 10-14 gm bleaching powder per liter of water, 70% ethanol, 4% formaldehyde, 70% isopropyl alcohol, 25% iodine or 6% hydrogen peroxide, solidification of liquid waste using dry super adsorbent polymers containing sanitizers or disinfecting agents like chlorine or glutaraldehyde, closed disposal systems, etc. Reference may be made to an article, “Liquid biomedical waste management: An emerging concern for physicians, Biswal S, Muller J Med Sci Res 2013, 4, 99-106 which states that the culture media containing high microbial loads or rich protein contents requires rigorous disinfection procedures, wherein inactivation is achieved using 5.23% sodium hypochlorite in a 1:10 dilution for a minimum of 8 hours inside a secured vessel followed by disposal down the sanitary sewer and subsequent flushing with a lot of cold water for at least of 10 minutes.

Solidification systems (super adsorbents) are deemed advantageous over other methods for the treatment and safer disposal of biomedical fluid wastes. Superabsorbent polymers are generally prepared by polymerizing unsaturated carboxylic acids or derivatives thereof, including, but not limited to, acrylic acid or its or metal/ammonium salts and alkyl acrylates, using an internal cross-linking agent such as oligo-functional monomers including, but not limited to, bisacrylamides, triacrylates, dimethacrylates, or triallylamines.

Several patents have educated the development of such solidification systems. Application Reference may be made to the U.S. Pat. No. 7,291,674B2 wherein surface cross-linked superabsorbent polymers with good liquid retention, permeability, and mechanical strength based on the absorbent structure.

Reference may be made to the U.S. Pat. No. 8,450,389B1, wherein one or a plurality of surface cross-linked superabsorbent particles in combination with a plurality of second particles for liquid solidification with reduced gel block and a method of solidifying liquid medical waste.

Reference may be made to the another patent U.S. Pat. No. 9,533,081B1, wherein a portable wound therapy system comprising a plurality of surface cross-linked superabsorbent particles along with a container, a wound covering, and a packet and included a similar liquid solidification system with reduced gel block.

Reference may be made to the U.S. Pat. No. 5,391,351A, wherein a body waste fluid solidification device comprising a hydrophilic xerogel of partially hydrolyzed poly (vinyl acetate), cross-linked poly (vinyl alcohol), cross-linked hydroxyalkyl acrylates and methacrylates, polymers and copolymers of ethylene oxide and polymers and copolymers acrylamide.

Reference may be made to the U.S. Pat. No. 6,797,857B2, wherein a solidifier for the solidification of a volume of liquid with a known density, comprising of three adsorbents with varying densities, thereby achieving controlled stabilization of a flowable material throughout its overall volume.

Reference may be made to the U.S. Pat. No. 5,424,265A, wherein a capsule for absorbing liquid waste with a powder adsorbent material disposed within said capsule, the body of the said capsule being water soluble leads to the adsorption of liquid waste located within a suction canister.

Reference may be made to the U.S. Pat. No. 9,102,806B2, wherein a particulate superabsorbent polymer capable of absorbing water, aqueous liquids, and blood, and a process to manufacture the said superabsorbent polymers. The said super adsorbent comprises of a 1-10 wt % of a thermoplastic polymer of any class selected from polyolefin, polyethylene, linear low density polyethylene, ethylene acrylic acid copolymer, styrene copolymers, ethylene alkyl methacrylate copolymer, polypropylene, ethylene vinyl acetate copolymer, polyamide, polyester, blends thereof, or copolymers thereof, where the surface is treated with a neutralized multivalent metal salt solution having a pH value similar to that of human skin.

Reference may be made to the U.S. Pat. No. 8,403,904B2, wherein a superabsorbent polymer comprising an internal cross-linking agent consisting of a silane derivative having a minimum of one vinyl group or one allyl group attached to a silicon atom, and at least one Si—O bond with high centrifuge retention capacity.

Super adsorbing polymers, methods for their preparation and application in liquid solidification have been described by several patents, viz, EP2739660B2, US20130310251A1, EP0273141B1, U.S. Pat. No. 8,476,189B1, JP5527916B2, U.S. Pat. No. 5,578,318A, DE69815670T2 and U.S. Pat. No. 8,821,363B1.

Solid wastes including, but not limited to, used cotton, tissue papers, syringes and needles are generally disinfected using approved disinfectants and/or sanitizers and are incinerated or recycled. Waste burial or land-fills, disposal in cemented pits, immobilization using plastic foam, sand, cement or clay, low/medium/high temperature burning, controlled incineration, steam autoclaving, rotary kiln, microwave treatment, chemical treatment, shredding, melting, etc. are the general practices in disposing solid waste.

(Reference may be made to WHO @ www.who.int/, and Medical Waste Management, International Committee of the Red Cross @ www.icrc.org/). A 1-10% solution of bleach, or hypochlorites, sodium hydroxide or other chemical disinfectants are used to disinfect biomedical waste. Heat, alkaline digesters and microwaves are also used for this purpose.

Acrylate based solidifiers, though cheap and vastly available, are not devoid of disadvantages. They generally take 10-15 minutes for complete gelation and are not easily recycled. Further, they are non-biodegradable and also some acrylates are shown to be flammable. Studies have indicated that several acrylates and their raw materials can be carcinogenic. Manufacturing of acrylics has both health and environmental impacts. Several chemicals used in the manufacturing as well as the chemical waste from acrylic plants are toxic. Hypochlorite (bleach) is not always effective with high organic content waste such as blood. Further, a disinfection system capable of instantaneously treating, immobilizing and disinfecting both liquid and solid medical wastes is not found in literature.

### Abbreviations Used

WHO: World Health Organization
min.: minutes
wt %: weight percentage
TiO₂: Titania/titanium dioxide
SiO₂: Silica/silicon dioxide
LaPO₄: Lanthanum phosphate
CePO₄: Cerium phosphate
NaOH: Sodium hydroxide
mg: milligram
mL: milliliter
kg: kilogram

### OBJECTIVES OF THE INVENTION

The main objective of the present invention is to provide a disinfecting composition which is capable of treating and disinfecting solid and fluid biomedical waste samples via instantaneous flocculation, gelation or solidification.

Another objective of the present invention is to provide a safer and cost-effective strategy for managing the biomedical wastes including solid and liquid wastes, via the reduction of spillage and occupational exposure.

Yet another objective of the present invention is to provide a process for the preparation of disinfecting composition for disposal of solid and fluid waste collected in a device at the required point of care.

Still another objective of the present invention is to provide a composition for the preparation for disposal of solid and fluid waste collected in a device via flocculation, gelation or solidification and further to destroy or at least disinfect or deactivate the infectious agents in the wastes for the preparation for disposal including treatment and transportation of the samples.

### SUMMARY OF THE INVENTION

This section describes the present invention in preferred embodiments.

In view of the above technical background, the present invention intends to disclose the development of a flocculant based gelation-solidification-disinfection composition for the treatment of biomedical waste.

Accordingly, the present invention provides a flocculant based disinfection composition comprising a solution A and a solution B wherein solution A is in a range of 0.1-2000 mg/mL, more preferably 1-200 mg/mL, 10-20% (v/v) of solution B; and the solution B is in a range of 0.1-700 mg/mL.

In an embodiment of the present invention, the solution A is poly-glutamic acid containing a base.

In another embodiment of the present invention, the base is sodium hydroxide.

In yet another embodiment of the present invention, the solution B is nanomaterial selected from the group consisting of oxides of titanium, aluminium (boehmite), silicon or phosphates of lanthanide elements.

In yet another embodiment of the present invention, the lanthanide is selected from cerium or lanthanum.

In still another embodiment of the present invention, the nanomaterial is in the range of 0.1-70 wt %.

In another aspect, the present invention provides a process for preparing the disinfection composition, comprising the steps of:
a. mixing poly-glutamic acid with sodium hydroxide and water to obtain a solution A;
b. preparing a solution B of an aqueous sol of nanomaterial;
c. adding a sample in solution B as obtained in step (b) followed by addition of solution A as obtained in step (a) to obtain the solution of disinfected composition;
   wherein the obtained solution is characterized as flocculated, gelled or solidified based on the concentration of the solution A.

In an embodiment of the present invention, the poly-glutamic acid used in step (a) is in the range of 10-20% (v/v) of solution B.

In another embodiment of the present invention, the sodium hydroxide used in step (a) is in the range of 0.1-2000 mg/mL, preferably 100-500 mg/mL of poly-glutamic acid.

In yet another embodiment of the present invention, the pH of the solution A is in the range of 9-13.

In yet another embodiment of the present invention, the sample used in step (c) is selected from the group consisting of salt, metal salt, aqueous waste, saliva, urine, blood or any solid sample, cotton, tissue, paper, needle, or swabs alone or in combination thereof.

In another aspect, the present invention provides a disinfection disposal device filled with the disinfected composition, the device comprising of:
a. an upper system [1];
b. a middle system [2];
c. a bottom system [3];
d. a screw cap [4] connected to the upper system;
e. a breakable screw-cap [5] connected between the upper and the bottom system.

In an embodiment of the present invention, the upper system is filled with the solution A.

In another embodiment of the present invention, the middle system is filled with the sample.

In yet another embodiment of the present invention, the bottom system is filled with the solution B.

In still another embodiment of the present invention, the sample is solid or liquid waste.

### BRIEF DESCRIPTION OF THE FIGURES

FIG. 1 illustrates the flocculation-gelation process when different volumes (10-100 μL) of polyglutamic acid are added to 1 mL of 2% TiO₂ sol in water. Instantaneous flocculation occurs in all samples, whereas instantaneous gelation occurs upon addition of 70 μL or larger amounts of polyglutamic acid (c=100 mg/mL containing 360 mg/mL NaOH).

FIG. 2 illustrates the gelation/solidification of 1 mL TiO₂ sol (2 wt %) in water mixed with aqueous waste upon addition of 200 μL polyglutamic acid (c=100 mg/mL containing 360 mg/mL NaOH).

FIG. 3 illustrates the gelation/solidification of 1 mL TiO₂ sol (2 wt %) in water mixed with excess sodium chloride (>300 mg) upon addition of 100 μL polyglutamic acid (c=100 mg/mL containing 360 mg/mL NaOH). Sodium chloride was added to TiO₂ sol in water before adding polyglutamic acid.

FIG. 4 illustrates the gelation/solidification of 1 mL TiO₂ sol (2 wt %) in water mixed with excess metal complex (iron bipyridine>100 mg) upon addition of 100 μL polyglutamic acid (c=100 mg/mL containing 360 mg/mL NaOH). The metal complex was added to TiO₂ sol in water before adding polyglutamic acid.

FIG. 5 illustrates the gelation/solidification of 1 mL TiO₂ sol (2 wt %) in water mixed with excess sodium chloride (>300 mg) and metal complex (iron bipyridine>100 mg) upon addition of 100 μL polyglutamic acid (c=100 mg/mL containing 360 mg/mL NaOH). Sodium chloride and the metal complex were added to TiO₂ sol in water before adding polyglutamic acid.

FIG. 6 illustrates the gelation/solidification of 1 mL TiO₂ sol (2 wt %) in water mixed with 6% BSA solution (1 mL in water) upon addition of 200 μL polyglutamic acid (c=100 mg/mL containing 360 mg/mL NaOH). BSA was added to TiO₂ sol in water before adding polyglutamic acid.

FIG. 7 illustrates the gelation/solidification of 1 mL artificial saliva. 200 μL polyglutamic acid (c=100 mg/mL containing 360 mg/mL NaOH) was added to 1 mL artificial saliva followed by 2 mL silica (SiO₂) sol (50 wt %) in water.

FIG. 8 illustrates the gelation/solidification of 0.75 mL artificial saliva. 2 mL boehmite (alumina) sol (10 wt %) in water was added to 0.75 mL artificial saliva followed by 200 μL polyglutamic acid (c=100 mg/mL containing 360 mg/mL NaOH).

FIG. 9 illustrates the gelation/solidification of 0.3-0.4 mL artificial saliva. 100 μL polyglutamic acid (c=100 mg/mL containing 360 mg/mL NaOH) was added to 0.3-0.4 mL artificial saliva followed by 1 mL TiO₂ sol (2 wt %) in water.

FIG. 10 illustrates the gelation/solidification of 1 mL artificial urine. 200 μL polyglutamic acid (c=100 mg/mL containing 360 mg/mL NaOH) was added to 1 mL artificial urine followed by 2 mL silica (SiO₂) sol (50 wt %) in water.

FIG. 11 illustrates the gelation/solidification of 1 mL artificial urine. 1.5 mL boehmite (alumina) sol (10 wt %) in water was added to 1 mL artificial urine followed by 200 μL polyglutamic acid (c=100 mg/mL containing 360 mg/mL NaOH).

FIG. 12 illustrates the gelation/solidification of 0.5 mL artificial blood. 200 μL polyglutamic acid (c=100 mg/mL containing 360 mg/mL NaOH) was added to 1 mL TiO₂ sol (2 wt %) in water containing 0.5 mL artificial blood.

FIG. 13 illustrates the immobilization of a solid swab. 5 mL TiO₂ sol (2 wt %) in water was added to a vial containing a swab, followed by 1 mL polyglutamic acid (c=100 mg/mL containing 360 mg/mL NaOH). The amount of the sol and the glutamic acid depends on the size of the solid sample.

FIG. 14 illustrates the immobilization of a solid swab. 5 mL SiO₂ sol (50 wt %) in water was added to a vial containing a swab, followed by 1 mL polyglutamic acid (c=100 mg/mL containing 360 mg/mL NaOH). The amount of the sol and the glutamic acid depends on the size of the solid sample.

FIG. 15 illustrates the flocculation of a solid swab. 12 mL TiO₂ sol (2 wt %) in water was added to a vial containing a swab, followed by 2 mL polyglutamic acid (c=100 mg/mL containing 360 mg/mL NaOH). The amount of the sol and the glutamic acid depends on the size of the solid sample.

FIG. 16 illustrates the immobilization of a needle. 5 mL SiO₂ sol (50 wt %) in water was added to a vial containing a needle, followed by 1 mL polyglutamic acid (c=100 mg/mL containing 360 mg/mL NaOH). The amount of the sol and the glutamic acid depends on the size of the solid sample.

FIG. 17 illustrates the immobilization of a needle. 5 mL boehmite sol (10 wt %) in water was added to a vial containing a needle, followed by 1 mL polyglutamic acid (c=100 mg/mL containing 360 mg/mL NaOH). The amount of the sol and the glutamic acid depends on the size of the solid sample.

FIG. 18 illustrates the immobilization of a needle. 5 mL LaPO₄ sol (2 wt %) in water was added to a vial containing a needle, followed by 1 mL polyglutamic acid (c=100 mg/mL containing 360 mg/mL NaOH). The amount of the sol and the glutamic acid depends on the size of the solid sample.

FIG. 19 illustrates the immobilization of cotton. 2 mL nanomaterial sol in water was added to a vial containing cotton, followed by 200 μL polyglutamic acid (c=100 mg/mL containing 360 mg/mL NaOH). 1: SiO₂ (50 wt %), 2:TiO₂ (2 wt %), 3: LaPO₄ (2 wt %), and 4: boehmite (10 wt %). The amount of the sol and the glutamic acid depends on the size of the solid sample.

FIG. 20 illustrates the immobilization of a piece of tissue paper. 2 mL nanomaterial sol in water was added to a vial containing a piece of tissue paper, followed by 200 μL polyglutamic acid (c=100 mg/mL containing 360 mg/mL NaOH). 1: SiO₂ (50 wt %), 2:TiO₂ (2 wt %), 3: LaPO₄ (2 wt %), and 4: boehmite (10 wt %). The amount of the sol and the glutamic acid depends on the size of the solid sample.

FIG. 21 illustrates the large scale solidification behavior. Photographs of (A) a 2000 mL glass beaker with ˜100 pieces of cotton, (B) 10% solution (10 g in 100 mL water) of polyglutamic acid (left) and 1000 mL 50% SiO₂ sol added to the 2000 mL beaker with ˜100 pieces of cotton (right), (C) immediately after addition and mixing of the two mixtures as in (B), (D) the solidified mixture as in (C) standing upside down, (E) the solidified mixture after complete mixing, (F) the solidified mixture after complete mixing as in (E) standing upside down, and (G) the solidified mixture with an added weight ˜2 kg, confirming its mechanical strength.

FIG. 22 illustrates the microbial disinfection in the treated samples (left) E. coli and (right) S. aureus.

FIG. 23 illustrates a prototype of an all-in-one sample collection-disinfection-disposal device for fluid samples, (A) consisting of three plastic collection vials mounted one on top of the other such that (B) the top vial contains polyglutamic acid solution (100 mg/mL with 360 mg sodium hydroxide per mL of the glutamic acid solution), the middle one for sample collection and the bottom one prefilled with the requisite amount of the nanomaterial (50 wt % SiO₂ is shown as an example) sol. The top compartment could be unscrewed and the samples could be collected in the middle compartment. Once collected sample is tested, the remaining sample could be flocculated, gelled or solidified by initially allowing (C) the sample to mix with the nanomaterial sol by breaking the junction between the middle and bottom compartments followed by (D) the addition of polyglutamic acid from the top compartment by breaking the junction between the top and middle compartments. The mixing of the three fluid mixtures allow for complete pathogenic disinfection.

FIG. 24 illustrates a prototype of an all-in-one sample collection-disinfection-disposal device for solid samples, (A) consisting of a plastic collection container for solid samples mounted on its top with another smaller plastic vial such that (B) the top vial contained polyglutamic acid solution (100 mg/mL with 360 mg sodium hydroxide per mL of the glutamic acid solution), and the bottom one was half-filled with the requisite amount of the nanomaterial (50 wt % SiO₂ is shown as an example) sol. (C) The top compartment could be unscrewed and the solid samples (cotton waste) could be collected in the bottom compartment and (D) could be flocculated, gelled or solidified by allowing the sample and the nanomaterial sol to mix with polyglutamic acid solution by breaking the junction between the two compartments. The mixing of the solutions and gelation allow for complete pathogenic disinfection.

FIG. 25: Design of the prototype of an all-in-one sample collection-disinfection-disposal device for fluid samples as shown in FIG. 23.

FIG. 26: Design of the prototype of an all-in-one sample collection-disinfection-disposal device for solid samples as shown in FIG. 24.

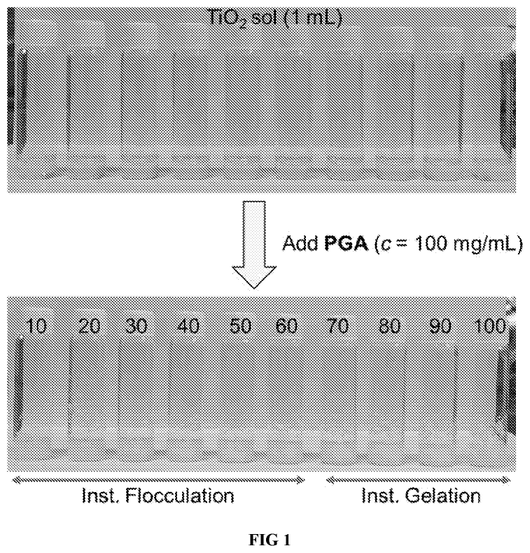

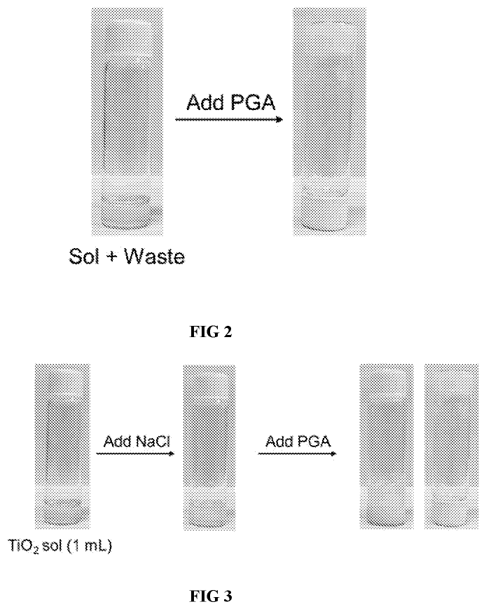

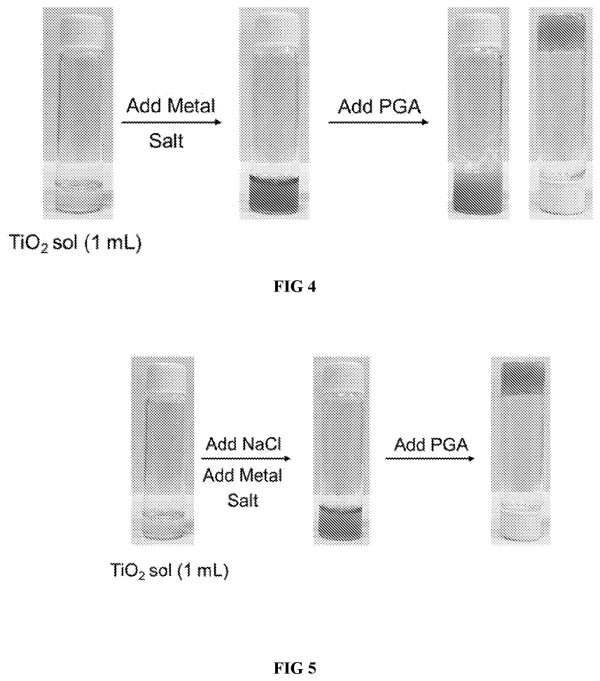

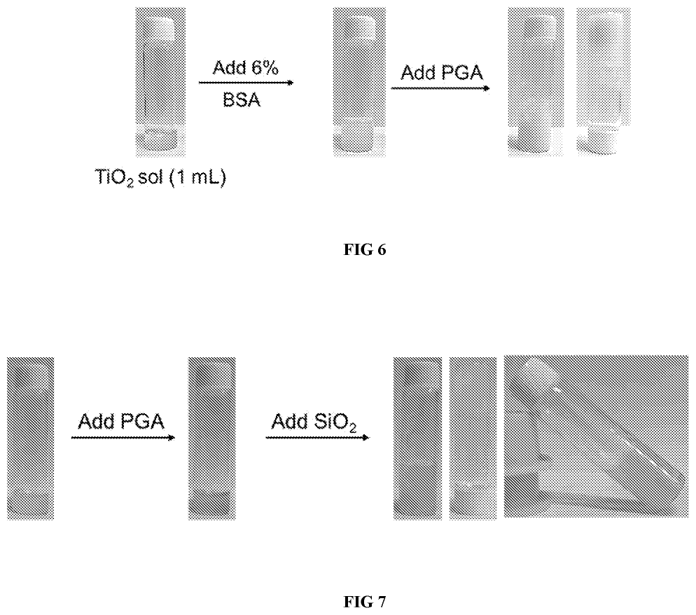

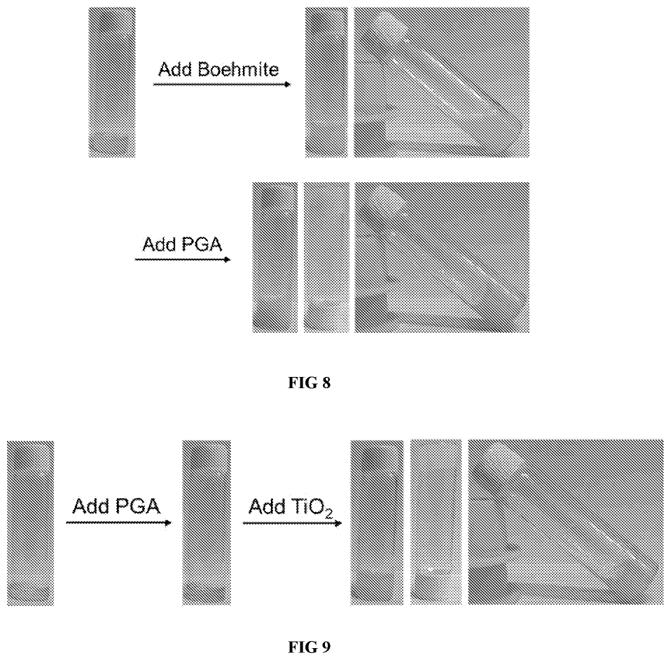

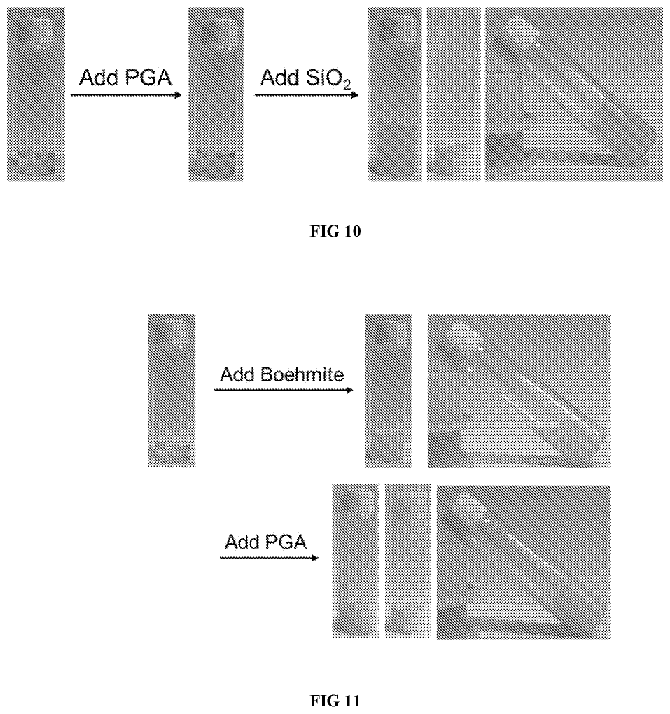

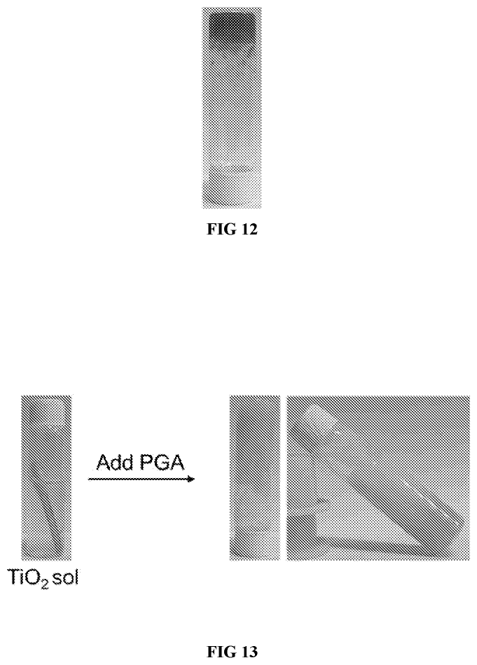

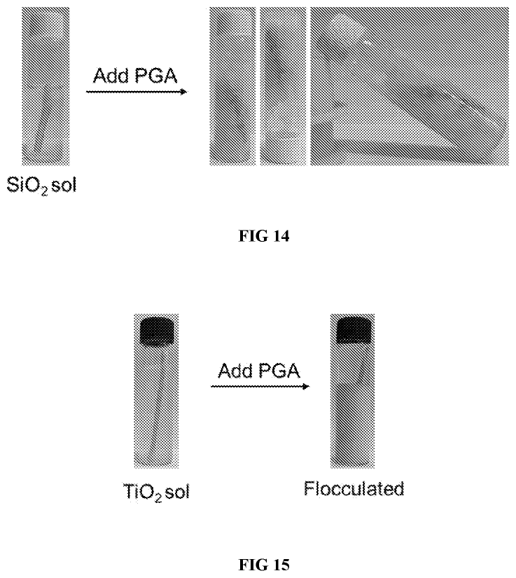

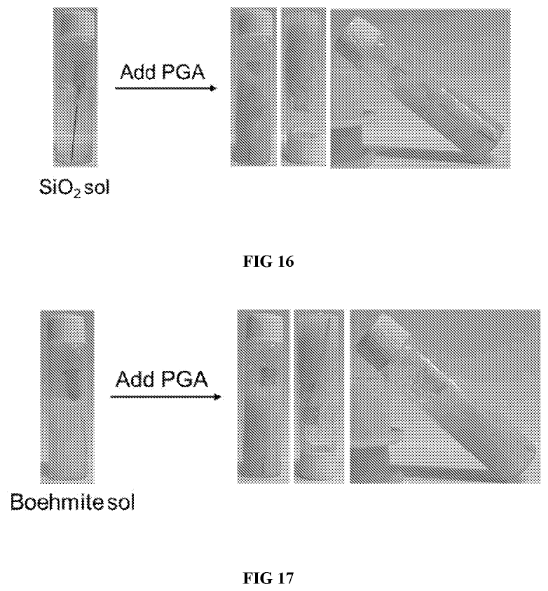

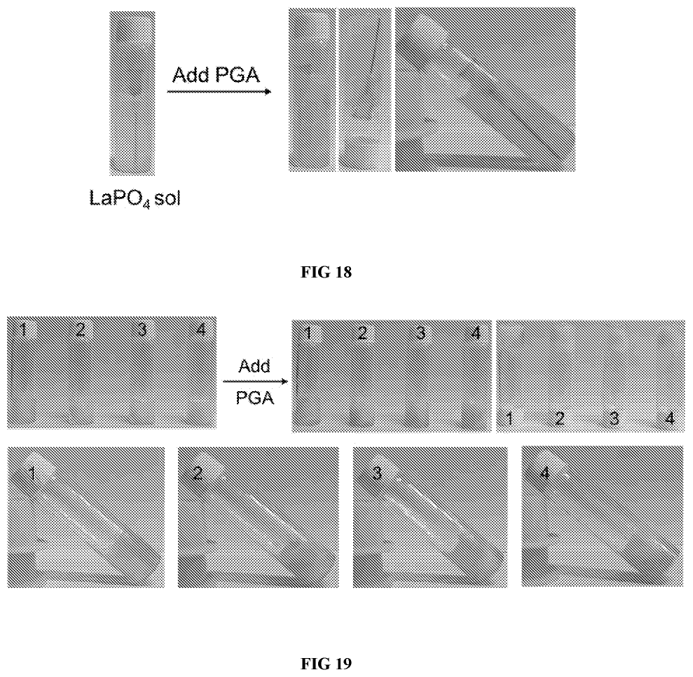

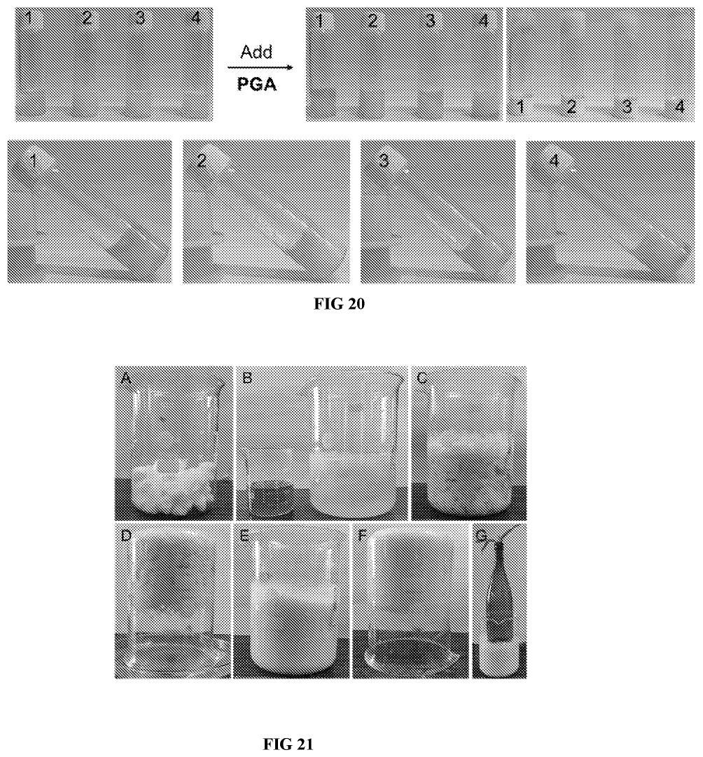

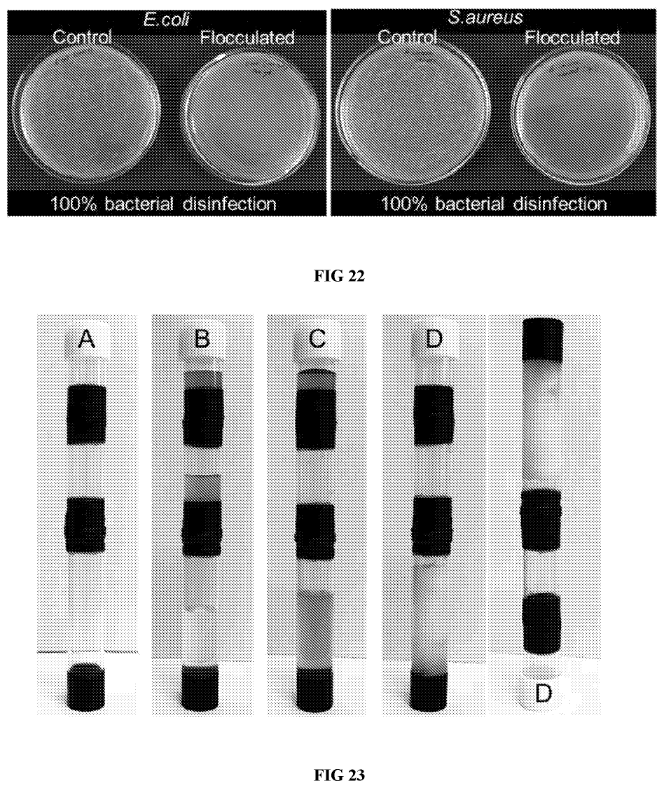

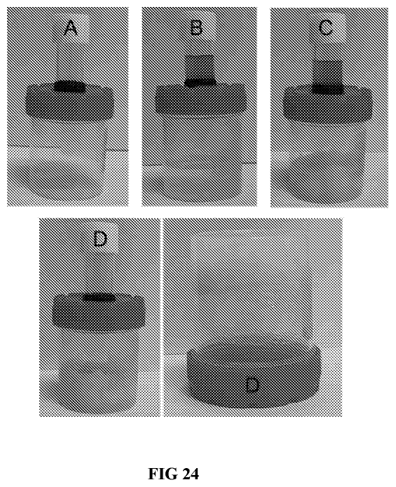

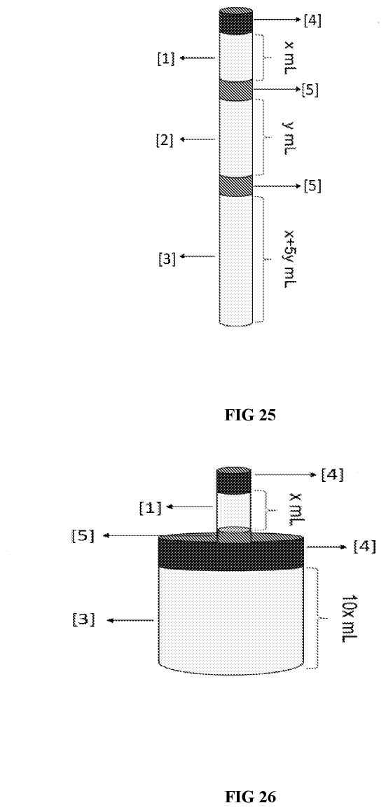

### DETAILED DESCRIPTION OF THE INVENTION

Accordingly, in the present invention the attached illustrations/drawings are intended for the purpose of describing and understanding invention in detail and not to limit the invention or its scope or both there.

In view of the above technical background, the present invention provides a flocculant based gelation-solidification-disinfection composition for the treatment of biomedical waste. The treatment composition described herein comprises of a selected nanomaterial as its sol in water and a poly-amino acid containing a basifying agent, which when mixed with solid or fluid waste samples at a defined volumetric and/or weighted composition leads to instantaneous flocculation/gelation/solidification with up to 100% microbial disinfection.

The main objective of the present invention is to provide a disinfection composition for the preparation for disposal of solid and fluid wastes collected in a collection vessel combined with the destruction, disinfection or deactivation of infectious agents including microorganisms such as, but not limited to, bacteria, fungus etc., viruses and other toxins, whereby the disposal including treatment, handling and transportation are deemed easier, safer and cost-effective.

Another aspect of the present invention provides a method to create a non-pourable environment for fluid medical wastes including, but not limited to saliva, urine, blood, etc. wherein risks related to spillage and occupational exposure are minimized, added with >99.9% microbial disfection.

In one aspect, the present subject matter is directed to the treatment of solid medical wastes including, but not limited to, cotton, tissue paper, swabs, needles, etc., wherein the risks related to accumulation of untreated and infected samples are minimized with >99.9% microbial disinfection.

Another aspect of the present invention discloses the volumetric composition of a sol of a defined nanomaterial selected from, but not limited to, oxides of titanium, aluminium, silicon or phosphates of lanthanide elements selected from, but not limited to, lanthanum or cerium in water at a predefined wt % with a biopolymer, specifically poly-amino acids, more specifically polyglutamic acid as its aqueous solution, containing a pH regulating base or alkali for complete disinfection of a predefined volume of fluid medical waste.

The invention also provides a method for the treatment of solid medical wastes including, but not limited to, cotton, swabs, needles or tissue paper, using a composition of a sol of a defined nanomaterial selected from, but not limited to, oxides of titanium, aluminium, silicon or phosphates of lanthanide elements selected from, but not limited to, lanthanum or cerium in water at a predefined wt % with a biopolymer, specifically poly-amino acids, more specifically polyglutamic acid as its aqueous solution, containing a pH regulating base or alkali, thereby rendering the samples non-infectious with >99.9% microbial disinfection.

Upon extensive investigations, the inventors of the present invention found that adding a poly-amino acid as its aqueous solution to a stable nanomaterial sol in water leads to instantaneous flocculation and the said flocculation process could further be controlled to effect gelation or solidification under carefully controlled conditions, depending on, but not limited to, the type of nanomaterial, concentration, volumetric ratio, etc.

The present invention provides a disinfection composition for the preparation for disposal of solid and fluid wastes collected in device at point of care, combined with the destruction, disinfection or deactivation of infectious agents including microorganisms such as, but not limited to, bacteria, fungus etc., viruses and other toxins, whereby the disposal including treatment, handling and transportation are deemed easier, safer and cost-effective. The addition of a flocculating agent to liquid waste reduces the risk of spills and aerosolization, whereas disinfection allows to dispose of the wastes thereof as non-regulated medical waste, which is less expensive than red-bagging. Segregation, transportation and incineration of such disinfected medical wastes are easier, safer and decrease medical waste disposal costs for a healthcare facility.

The present invention provides a volumetric composition of a sol of a defined nanomaterial selected from, but not limited to, oxides of titanium, aluminium, silicon or phosphates of lanthanide elements selected from, but not limited to, lanthanum or cerium in water at a predefined wt %, preferably 0.1-50 wt % titania (TiO₂) sol in water, more preferably 1-5 wt % titania (TiO₂) sol in water, preferably 0.1-60 wt % boehmite (alumina) sol in water, more preferably 5-10 wt % boehmite (alumina) sol in water, preferably 0.1-50 wt % lanthanum or cerium phosphate (LaPO₄ or CePO₄) sol in water, more preferably 1-5 wt % lanthanum or cerium phosphate (LaPO₄ or CePO₄) sol in water, preferably 0.1-70 wt % silica (SiO₂) in water, more preferably 25-50 wt % silica (SiO₂) sol in water with a biopolymer, specifically poly-amino acids, more specifically polyglutamic acid as its aqueous solution, at a preferred concentration of 0.1-2000 mg/mL, more preferably at a concentration of 1-200 mg/mL, containing a pH regulating base or alkali, the said base is hydroxides of alkali metals or alkaline earth metals, basic salts of metals and organic cations, more preferably sodium hydroxide at a concentration of 0.1-2000 mg per mL of the poly-amino acid, more preferably 100-500 mg per mL of the poly-amino acid, the said mixture leading to a complete disinfection of a predefined volume of fluid medical waste.

The present invention provides a self-disinfecting flocculant-based gelation-solidification composition for the treatment and disposal of biomedical waste. The treatment composition disclosed herein comprises of a poly-amino acid as its aqueous solution, the said solution basified to an alkaline pH>9 using a base, preferably pH>11, more preferably pH>13, the said base is preferably sodium hydroxide, and a selected nanomaterial as its sol in water, which when subjected to mixing with solid or fluid waste samples at a defined volumetric and/or weighted composition leads to instantaneous flocculation/gelation/solidification with up to 100% microbial disinfection.

The present invention provides a process for the treatment of solid medical wastes including, but not limited to, cotton, swabs, needles or tissue paper, using a composition of a sol of a defined nanomaterial selected from, but not limited to, oxides of titanium, aluminium, silicon or phosphates of lanthanide elements selected from, but not limited to, lanthanum or cerium in water at a predefined wt %, preferably 0.1-50 wt % titania (TiO₂) sol in water, more preferably 1-wt % titania (TiO₂) sol in water, preferably 0.1-60 wt % boehmite (alumina) sol in water, more preferably 5-10 wt % boehmite (alumina) sol in water, preferably 0.1-50 wt % lanthanum or cerium phosphate (LaPO₄ or CePO₄) sol in water, more preferably 1-5 wt % lanthanum or cerium phosphate (LaPO₄ or CePO₄) sol in water, preferably 0.1-70 wt % silica (SiO₂) in water, more preferably 25-50 wt % silica (SiO₂) sol in water, with a biopolymer, specifically poly-amino acids, more specifically polyglutamic acid as its aqueous solution, at a preferred concentration of 0.1-2000 mg/mL, more preferably at a concentration of 1-200 mg/mL, containing a pH regulating base or alkali, the said base is hydroxides of alkali metals or alkaline earth metals, basic salts of metals and organic cations, more preferably sodium hydroxide at a concentration of 0.1-2000 mg per mL of the poly-amino acid, more preferably 100-500 mg per mL of the poly-amino acid, thereby rendering the samples non-infectious with >99.9% microbial disinfection.

The present invention provides the disinfection composition, the composition comprising of the sol of one or a plurality of nanomaterials and pH regulated poly-amino acid, specifically polyglutamic acid, as its aqueous solution, at a preferred concentration of 0.1-2000 mg/mL, more preferably at a concentration of 1-200 mg/mL, containing a pH regulating base or alkali, the said base is hydroxides of alkali metals or alkaline earth metals, basic salts of metals and organic cations, more preferably sodium hydroxide at a concentration of 0.1-2000 mg per mL of the poly-amino acid, more preferably 100-500 mg per mL of the poly-amino acid, with effective flocculation/gelation/solidification of solid of fluid samples containing proteins, microbial cultures, salt or metal ions in high concentrations.

The present invention provides a method to create a non-pourable environment for free-flowing fluid medical wastes including, but not limited to saliva, urine, blood, etc. wherein risks related to spillage and occupational exposure are minimized, added with >99.9% microbial disfection. Samples of body fluids were simulated with spikes of high protein content, salt or sugar as described in the respective examples.

The other vital constitutional element in the present invention relates to the treatment of solid medical wastes including, but not limited to, cotton, tissue paper, swabs, needles, etc., that may also lead to spread of infections and simple absorbers or hypochlorites that are currently in use are not always capable of treating such solid wastes, wherein the risks related to accumulation of untreated and infected samples are minimized with >99.9% microbial disinfection.

Another aspect of the present invention is directed to creating all-in-one sample collection-solidification-disinfection device of requisite dimensions capable of collecting the solid or liquid sample, flocculating/gelating/solidifying the samples as and when required and disinfecting the same for preparation for its disposal.

### EXAMPLES

Following examples are given by way of illustration and therefore should not be construed to limit the scope of the invention.

### Example 1. Flocculation-Gelation of TiO₂ Sol Using Polyglutamic Acid

A stock solution of polyglutamic acid at a concentration of 100 mg/mL was prepared in water and sodium hydroxide (360 mg per mL of polyglutamic acid solution) was added. 1 mL TiO₂ sol (2 wt %) in water was pipetted out into ten 8 mL glass vials. 10-100 μL polyglutamic acid solution was added to each of the above vials and mixed. Flocculation was found to occur in all the vial immediately upon mixing, whereas instantaneous gelation occurs upon addition of 70 μL or larger amounts of polyglutamic acid (c=100 mg/mL containing 360 mg/mL NaOH).

### Example 2. Flocculation-Gelation of Aqueous Waste in TiO₂ Sol Using Polyglutamic Acid

A stock solution of polyglutamic acid at a concentration of 100 mg/mL was prepared in water and sodium hydroxide (360 mg per mL of polyglutamic acid solution) was added. 1 mL TiO₂ sol (2 wt %) in water was added to 1 mL aqueous waste in an 8 mL glass vial. 200 μL polyglutamic acid solution was added to the above vial and mixed. Instantaneous gelation occurred upon addition of polyglutamic acid (c=100 mg/mL containing 360 mg/mL NaOH).

### Example 3. Gelation of TiO₂ Sol Containing Excess Sodium Chloride Using Polyglutamic Acid

A stock solution of polyglutamic acid at a concentration of 100 mg/mL was prepared in water and sodium hydroxide (360 mg per mL of polyglutamic acid solution) was added. Excess sodium chloride (300 mg) was added to 1 mL TiO₂ sol (2 wt %) in water in an 8 mL glass vial. 200 μL polyglutamic acid solution was added to the above vial and mixed. Instantaneous gelation occurred upon addition of polyglutamic acid (c=100 mg/mL containing 360 mg/mL NaOH).

### Example 4. Gelation of TiO₂ Sol Containing Excess Metal Salts Using Polyglutamic Acid

A stock solution of polyglutamic acid at a concentration of 100 mg/mL was prepared in water and sodium hydroxide (360 mg per mL of polyglutamic acid solution) was added. Fe(II)-bipyridine complex (100 mg, Fe(bpy)₃Cl₂) was added to 1 mL TiO₂ sol (2 wt %) in water in an 8 mL glass vial. 200 μL polyglutamic acid solution was added to the above vial and mixed. Instantaneous gelation occurred upon addition of polyglutamic acid (c=100 mg/mL containing 360 mg/mL NaOH).

### Example 5. Gelation of TiO₂ Sol Containing Excess Sodium Chloride and Metal Salts Using Polyglutamic Acid

A stock solution of polyglutamic acid at a concentration of 100 mg/mL was prepared in water and sodium hydroxide (360 mg per mL of polyglutamic acid solution) was added. Excess sodium chloride (300 mg) was added to 1 mL TiO₂ sol (2 wt %) in water in an 8 mL glass vial, followed by Fe(II)-bipyridine complex (100 mg, Fe(bpy)₃Cl₂). 200 μL polyglutamic acid solution was added to the above vial and mixed. Instantaneous gelation occurred upon addition of polyglutamic acid (c=100 mg/mL containing 360 mg/mL NaOH).

### Example 6. Gelation of TiO₂ Sol with Protein Content Mimicing Human Blood Using Polyglutamic Acid

A stock solution of polyglutamic acid at a concentration of 100 mg/mL was prepared in water and sodium hydroxide (360 mg per mL of polyglutamic acid solution) was added. Bovine serum albumin (BSA, 6% in water mimicing human blood, 1 mL) was added to 1 mL TiO₂ sol (2 wt %) in water in an 8 mL glass vial. 200 μL polyglutamic acid solution was added to the above vial and mixed. Instantaneous gelation occurred upon addition of polyglutamic acid (c=100 mg/mL containing 360 mg/mL NaOH).

### Example 7. Gelation-Solidification of SiO₂ Sol Using Polyglutamic Acid

A stock solution of polyglutamic acid at a concentration of 100 mg/mL was prepared in water and sodium hydroxide (360 mg per mL of polyglutamic acid solution) was added. 100 μL polyglutamic acid solution was added to 1 mL SiO₂ Sol (50 wt % in water) in an 8 mL glass vial and mixed. Instantaneous gelation leading to solidification occurred upon addition of polyglutamic acid (c=100 mg/mL containing 360 mg/mL NaOH).

### Example 8. Gelation-Solidification of Aqueous Waste in SiO₂ Sol Using Polyglutamic Acid

A stock solution of polyglutamic acid at a concentration of 100 mg/mL was prepared in water and sodium hydroxide (360 mg per mL of polyglutamic acid solution) was added. 1 mL SiO₂ sol (50 wt %) in water was added to 1 mL aqueous waste in an 8 mL glass vial. 200 μL polyglutamic acid solution was added to the above vial and mixed. Instantaneous gelation leading to solidification occurred upon addition of polyglutamic acid (c=100 mg/mL containing 360 mg/mL NaOH).

### Example 9. Gelation-Solidification of TiO₂ Sol Containing Excess Metal Salts Using Polyglutamic Acid

A stock solution of polyglutamic acid at a concentration of 100 mg/mL was prepared in water and sodium hydroxide (360 mg per mL of polyglutamic acid solution) was added. Fe(II)-bipyridine complex (100 mg, Fe(bpy)₃Cl₂) was added to 1 mL SiO₂ sol (50 wt %) in water in an 8 mL glass vial. 200 μL polyglutamic acid solution was added to the above vial and mixed. Instantaneous gelation leading to solidification occurred upon addition of polyglutamic acid (c=100 mg/mL containing 360 mg/mL NaOH).

### Example 10. Gelation-Solidification of SiO₂ Sol with Protein Content Mimicing Human Blood Using Polyglutamic Acid

A stock solution of polyglutamic acid at a concentration of 100 mg/mL was prepared in water and sodium hydroxide (360 mg per mL of polyglutamic acid solution) was added. Bovine serum albumin (BSA, 6% in water mimicing human blood, 1 mL) was added to 1 mL SiO₂ sol in water in an 8 mL glass vial. 200 μL polyglutamic acid solution was added to the above vial and mixed. Instantaneous gelation followed by solidification occurred upon addition of polyglutamic acid (c=100 mg/mL containing 360 mg/mL NaOH).

### Example 11. Flocculation-Gelation of Boehmite (Alumina) Sol Using Polyglutamic Acid

A stock solution of polyglutamic acid at a concentration of 100 mg/mL was prepared in water and sodium hydroxide (360 mg per mL of polyglutamic acid solution) was added. 100 μL polyglutamic acid solution was added to 1 mL Boehmite Sol (10 wt % in water) in an 8 mL glass vial and mixed. Instantaneous flocculation leading to gelation occurred upon addition of polyglutamic acid (c=100 mg/mL containing 360 mg/mL NaOH).

### Example 12. Flocculation-Gelation of Aqueous Waste in Boehmite (Alumina) Sol Using Polyglutamic Acid

A stock solution of polyglutamic acid at a concentration of 100 mg/mL was prepared in water and sodium hydroxide (360 mg per mL of polyglutamic acid solution) was added. 1 mL Boehmite Sol (10 wt %) in water was added to 1 mL aqueous waste in an 8 mL glass vial. 200 μL polyglutamic acid solution was added to the above vial and mixed. Instantaneous flocculation leading to gelation occurred upon addition of polyglutamic acid (c=100 mg/mL containing 360 mg/mL NaOH).

### Example 13. Flocculation of Boehmite (Alumina) Sol with Protein Content Mimicing Human Blood Using Polyglutamic Acid

A stock solution of polyglutamic acid at a concentration of 100 mg/mL was prepared in water and sodium hydroxide (360 mg per mL of polyglutamic acid solution) was added. Bovine serum albumin (BSA, 6% in water mimicing human blood, 1 mL) was added to 1 mL SiO₂ sol in water in an 8 mL glass vial. 100 μL polyglutamic acid solution was added to the above vial and mixed. Instantaneous flocculation occurred upon addition of polyglutamic acid (c=100 mg/mL containing 360 mg/mL NaOH).

### Example 14. Flocculation-Gelation of Boehmite (Alumina) Sol with Protein Content Mimicing Human Blood Using Polyglutamic Acid

A stock solution of polyglutamic acid at a concentration of 100 mg/mL was prepared in water and sodium hydroxide (360 mg per mL of polyglutamic acid solution) was added. Bovine serum albumin (BSA, 6% in water mimicing human blood, 1 mL) was added to 1 mL SiO₂ sol in water in an 8 mL glass vial. 200 μL polyglutamic acid solution was added to the above vial and mixed. Instantaneous flocculation leading to gelation occurred upon addition of polyglutamic acid (c=100 mg/mL containing 360 mg/mL NaOH).

### Example 15. Flocculation-Gelation of LnPO₄ (Ln=La, Ce) Sol using Polyglutamic acid

A stock solution of polyglutamic acid at a concentration of 100 mg/mL was prepared in water and sodium hydroxide (360 mg per mL of polyglutamic acid solution) was added. 100 μL polyglutamic acid solution was added to 1 mL LaPO₄ Sol (2 wt % in water) in an 8 mL glass vial and mixed. Instantaneous flocculation leading to gelation occurred upon addition of polyglutamic acid (c=100 mg/mL containing 360 mg/mL NaOH).

### Example 16. Flocculation-Gelation of Aqueous Waste in LnPO₄ (Ln=La, Ce) Sol Using Polyglutamic Acid

A stock solution of polyglutamic acid at a concentration of 100 mg/mL was prepared in water and sodium hydroxide (360 mg per mL of polyglutamic acid solution) was added. 1 mL LaPO₄ sol (2 wt %) in water was added to 1 mL aqueous waste in an 8 mL glass vial. 200 μL polyglutamic acid solution was added to the above vial and mixed. Instantaneous flocculation leading to gelation occurred upon addition of polyglutamic acid (c=100 mg/mL containing 360 mg/mL NaOH).

### Example 17. Preparation of Artificial Saliva

Artificial saliva was prepared according to the following two procedures: (i) Mixing 1.5 mM Ca(NO₃)₂, 0.90 mM KH₂PO₄, 130 mM KCl and 60 mM Tris buffer at pH 7.4 (Reference may be made to: Kirkham, J.; et al., Self-assembling peptide scaffolds promote enamel remineralization, J. Dental Res. 2007, 86, 426-430). (ii) Mixing sodium chloride (0.06 g), potassium chloride (0.072 g), calcium chloride dihydrate (0.022 g), potassium dihydrogen phosphate (0.068 g), disodium hydrogen phosphate dodecahydrate (0.086 g), potassium thiocyanate (0.006 g), sodium hydrogen carbonate (0.15 g), and citric acid (0.003 g) in 100 mL distilled water at pH 6.5 (Reference may be made to: Duffó, G. S.; et al., Development of an artificial saliva solution for studying the corrosion behavior of dental alloys. Corrosion 2004, 60, 594-602).

### Example 18. Flocculation-Gelation of Artificial Saliva in SiO₂ Sol Using Polyglutamic Acid

A stock solution of polyglutamic acid at a concentration of 100 mg/mL was prepared in water and sodium hydroxide (360 mg per mL of polyglutamic acid solution) was added. 1 mL artificial saliva was taken in an 8 mL glass vial and 200 μL polyglutamic acid solution was added and mixed. 2 mL SiO₂ sol (50 wt %) in water was then added and instantaneous flocculation leading to gelation occurred upon mixing.

### Example 19. Gelation-Solidification of Artificial Saliva in Boehmite (Alumina) Sol Using Polyglutamic Acid

A stock solution of polyglutamic acid at a concentration of 100 mg/mL was prepared in water and sodium hydroxide (360 mg per mL of polyglutamic acid solution) was added. 0.75 mL artificial saliva was taken in an 8 mL glass vial and 2 mL Boehmite sol (10 wt %) in water was added. 200 μL polyglutamic acid solution was then added and instantaneous gelation leading solidification to occurred upon mixing.

### Example 20. Flocculation-Gelation of Artificial Saliva in TiO₂ Sol Using Polyglutamic Acid

A stock solution of polyglutamic acid at a concentration of 100 mg/mL was prepared in water and sodium hydroxide (360 mg per mL of polyglutamic acid solution) was added. 0.3-0.4 mL artificial saliva was taken in an 8 mL glass vial and 1 mL TiO₂ sol (2 wt %) in water was added. 100 μL polyglutamic acid solution was then added and instantaneous flocculation leading to gelation occurred upon mixing.

### Example 21. Preparation of Artificial Urine

To 75 mL of distilled water in a container, urea (1.82 g) was added and shaken well to dissolve. Sodium chloride (0.75 g), potassium chloride (0.45 g) and sodium phosphate (0.48 g) were further added to the above mixture and mixed well until dissolved. The pH was adjusted to be between 5 and 7. Creatinine (200 mg) and albumin powder (5 mg) were added and mixed gently. The artificial urine thus obtained was further spiked with a few mg of glucose before each experiment.

### Example 22. Gelation-Solidification of Artificial Urine in SiO₂ Sol Using Polyglutamic Acid

A stock solution of polyglutamic acid at a concentration of 100 mg/mL was prepared in water and sodium hydroxide (360 mg per mL of polyglutamic acid solution) was added. 1 mL glucose spiked artificial urine was taken in an 8 mL glass vial and 200 μL polyglutamic acid solution was added and mixed. 2 mL SiO₂ sol (50 wt %) in water was then added and instantaneous gelation leading to solidification occurred upon mixing.

### Example 23. Flocculation-Gelation of Artificial Urine in Boehmite (Alumina) Sol Using Polyglutamic Acid

A stock solution of polyglutamic acid at a concentration of 100 mg/mL was prepared in water and sodium hydroxide (360 mg per mL of polyglutamic acid solution) was added. 1 mL glucose spiked artificial urine was taken in an 8 mL glass vial and 1.5 mL Boehmite sol (10 wt %) in water was added. 200 μL polyglutamic acid solution was then added and instantaneous flocculation leading to gelation occurred upon mixing.

### Example 24. Preparation of Artificial Blood

A 6% solution of BSA was prepared in distilled water. A small amount of red water soluble dye was then added to impart color.

### Example 25. Flocculation-Gelation of Artificial Blood in TiO₂ Sol Using Polyglutamic Acid

A stock solution of polyglutamic acid at a concentration of 100 mg/mL was prepared in water and sodium hydroxide (360 mg per mL of polyglutamic acid solution) was added. 0.5 mL artificial saliva was taken in an 8 mL glass vial and 1 mL TiO₂ sol (2 wt %) in water was added. 200 μL polyglutamic acid solution was then added and instantaneous flocculation leading to gelation occurred upon mixing.

### Example 26. Gelation-Solidification of Artificial Blood in SiO₂ Sol Using Polyglutamic Acid

A stock solution of polyglutamic acid at a concentration of 100 mg/mL was prepared in water and sodium hydroxide (360 mg per mL of polyglutamic acid solution) was added. 0.5 mL artificial blood was taken in an 8 mL glass vial and 200 μL polyglutamic acid solution was added and mixed. 1 mL SiO₂ sol (50 wt %) in water was then added and instantaneous gelation leading to solidification occurred upon mixing.

### Example 27. Immobilization of a Solid Swab in TiO₂ Sol Gelled Using Polyglutamic Acid

A piece of swab (4 cm) was taken in an 8 mL glass vial. 5 mL TiO₂ sol (2 wt %) in water was added to the vial (so that the swab was almost immersed in the sol), followed by 1 mL polyglutamic acid (c=100 mg/mL containing 360 mg/mL NaOH). Instantaneous flocculation leading to gelation occurred upon mixing and the swab was immobilized in the solid matrix. The amount of the sol and the glutamic acid depends on the size of the solid sample.

### Example 28. Immobilization of a Solid Swab in SiO₂ Sol Gelled Using Polyglutamic Acid

A piece of swab (4 cm) was taken in an 8 mL glass vial. 5 mL SiO₂ sol (50 wt %) in water was added to the vial (so that the swab was almost immersed in the sol), followed by 1 mL polyglutamic acid (c=100 mg/mL containing 360 mg/mL NaOH). Instantaneous gelation leading to solidification occurred upon mixing and the swab was immobilized in the solid matrix. The amount of the sol and the glutamic acid depends on the size of the solid sample.

### Example 29. Treatment of a Solid Swab in TiO₂ Sol Flocculated Using Polyglutamic Acid

A piece of swab (8 cm) was taken in a 15 mL glass vial. 12 mL TiO₂ sol (2 wt %) in water was added to the vial (so that the swab was almost immersed in the sol), followed by 2 mL polyglutamic acid (c=100 mg/mL containing 360 mg/mL NaOH). Instantaneous flocculation leading to separation of solid and liquid components occurred upon mixing. The amount of the sol and the glutamic acid depends on the size of the solid sample.

### Example 30. Immobilization of a Syringe Needle in SiO₂ Sol Gelled Using Polyglutamic Acid

A needle (4 cm) was taken in an 8 mL glass vial. 5 mL SiO₂ sol (50 wt %) in water was added to the vial (so that the needle was almost immersed in the sol), followed by 1 mL polyglutamic acid (c=100 mg/mL containing 360 mg/mL NaOH). Instantaneous gelation leading to solidification occurred upon mixing and the needle was immobilized in the solid matrix. The amount of the sol and the glutamic acid depends on the size of the solid sample.

### Example 31. Immobilization of a Syringe Needle in Boehmite (Alumina) Sol Gelled Using Polyglutamic Acid

A needle (4 cm) was taken in an 8 mL glass vial. 5 mL boehmite sol (10 wt %) in water was added to the vial (so that the needle was almost immersed in the sol), followed by 1 mL polyglutamic acid (c=100 mg/mL containing 360 mg/mL NaOH). Instantaneous flocculation leading to gelation occurred upon mixing and the needle was immobilized in the solid matrix. The amount of the sol and the glutamic acid depends on the size of the solid sample.

### Example 32. Immobilization of a Syringe Needle in LaPO₄ Sol Gelled Using Polyglutamic Acid

A needle (4 cm) was taken in an 8 mL glass vial. 5 mL LaPO₄ sol (2 wt %) in water was added to the vial (so that the needle was almost immersed in the sol), followed by 1 mL polyglutamic acid (c=100 mg/mL containing 360 mg/mL NaOH). Instantaneous flocculation leading to gelation occurred upon mixing and the needle was immobilized in the solid matrix. The amount of the sol and the glutamic acid depends on the size of the solid sample.

### Example 33. Immobilization of Cotton Waste in SiO₂ Sol Gelled Using Polyglutamic Acid

A piece of cotton waste was taken in an 8 mL glass vial. 2 mL SiO₂ sol (50 wt %) in water was added to the vial (so that the cotton was almost immersed in the sol), followed by 200 μL polyglutamic acid (c=100 mg/mL containing 360 mg/mL NaOH). Instantaneous gelation leading to solidification occurred upon mixing and the cotton waste was immobilized in the solid matrix. The amount of the sol and the glutamic acid depends on the size of the solid sample.

### Example 34. Immobilization of Cotton Waste in TiO₂ Sol Flocculated Using Polyglutamic Acid

A piece of cotton waste was taken in an 8 mL glass vial. 2 mL TiO₂ sol (2 wt %) in water was added to the vial (so that the cotton was almost immersed in the sol), followed by 200 μL polyglutamic acid (c=100 mg/mL containing 360 mg/mL NaOH). Instantaneous flocculation leading to gelation occurred upon mixing and the cotton waste was immobilized in the solid matrix. The amount of the sol and the glutamic acid depends on the size of the solid sample.

### Example 35. Immobilization of Cotton Waste in LaPO₄ Sol Gelled Using Polyglutamic Acid

A piece of cotton waste was taken in an 8 mL glass vial. 2 mL LaPO₄ sol (2 wt %) in water was added to the vial (so that the needle was almost immersed in the sol), followed by 200 μL polyglutamic acid (c=100 mg/mL containing 360 mg/mL NaOH). Instantaneous flocculation leading to gelation occurred upon mixing and the cotton waste was immobilized in the solid matrix. The amount of the sol and the glutamic acid depends on the size of the solid sample.

### Example 36. Immobilization of Cotton Waste in Boehmite (Alumina) Sol Gelled Using Polyglutamic Acid

A piece of cotton waste was taken in an 8 mL glass vial. 2 mL boehmite sol (10 wt %) in water was added to the vial (so that the cotton was almost immersed in the sol), followed by 1 mL polyglutamic acid (c=100 mg/mL containing 360 mg/mL NaOH). Instantaneous flocculation leading to gelation occurred upon mixing and the cotton waste was immobilized in the solid matrix. The amount of the sol and the glutamic acid depends on the size of the solid sample.

### Example 37. Immobilization of Tissue Paper in SiO₂ Sol Gelled Using Polyglutamic Acid

A piece of tissue paper was taken in an 8 mL glass vial. 2 mL SiO₂ sol (50 wt %) in water was added to the vial (so that the tissue paper was almost immersed in the sol), followed by 200 □L polyglutamic acid (c=100 mg/mL containing 360 mg/mL NaOH). Instantaneous gelation leading to solidification occurred upon mixing and the tissue paper was immobilized in the solid matrix. The amount of the sol and the glutamic acid depends on the size of the solid sample.

### Example 38. Immobilization of Tissue Paper in TiO₂ Sol Flocculated Using Polyglutamic Acid

A piece of tissue paper was taken in an 8 mL glass vial. 2 mL TiO₂ sol (2 wt %) in water was added to the vial (so that the tissue paper was almost immersed in the sol), followed by 200 □L polyglutamic acid (c=100 mg/mL containing 360 mg/mL NaOH). Instantaneous flocculation leading to gelation occurred upon mixing and the tissue paper was immobilized in the solid matrix. The amount of the sol and the glutamic acid depends on the size of the solid sample.

### Example 39. Immobilization of Tissue Paper in LaPO₄ Sol Gelled Using Polyglutamic Acid

A piece of tissue paper was taken in an 8 mL glass vial. 2 mL LaPO₄ sol (2 wt %) in water was added to the vial (so that the tissue paper was almost immersed in the sol), followed by 200 μL polyglutamic acid (c=100 mg/mL containing 360 mg/mL NaOH). Instantaneous flocculation leading to gelation occurred upon mixing and the tissue paper was immobilized in the solid matrix. The amount of the sol and the glutamic acid depends on the size of the solid sample.

### Example 40. Immobilization of Tissue Paper in Boehmite (Alumina) Sol Gelled Using Polyglutamic Acid

A piece of tissue paper was taken in an 8 mL glass vial. 2 mL boehmite sol (10 wt %) in water was added to the vial (so that the tissue paper was almost immersed in the sol), followed by 1 mL polyglutamic acid (c=100 mg/mL containing 360 mg/mL NaOH). Instantaneous flocculation leading to gelation occurred upon mixing and the tissue paper was immobilized in the solid matrix. The amount of the sol and the glutamic acid depends on the size of the solid sample.

### Example 41. Antimicrobial Studies

Cultures of Escherichia coli and Staphylococcus aureus were prepared in Luria Bertiani (LB) medium and taken for test at 18 hours old stage where the colony forming units (cfus) are approximately 1-3×10⁶ per millilitre for E. coli or S. aureus. (previously standardized based on optical densities at 600 nm). 1 mL of the nanomaterial (2 wt % TiO₂, 50 wt % SiO₂, 2 wt % LaPO₄ or 10 wt % boehmite) sol in water was spiked with 0.5 to 1.0 mL of the bacterial suspension (spiking solution) and mixed by swirling the bottle. Aqueous Polyglutamic acid solution (100 mg/mL, containing 360 mg sodium hydroxide per mL of the polyglutamic acid solution) was added such that its concentration is 10% of the total volume (including spiking solution), when the whole mixture becomes a gel. The gel is mixed well and diluted 10× in sterile saline and 100 μL of the diluted solution was plated onto LB agar plates and incubated over night at 37° C. Parallely, the original bacterial suspension was diluted serially in sterile saline and 100 μL of the appropriate dilutions were plated on LB agar plates and incubated as for the test sample that served as controls. Colonies were counted the next day and based on applied dilution, the number of CFUs/mL of the original bacterial suspension added to the sol and the CFUs in the gelled disinfectant were calculated. Efficiency was calculated as follows: [(No. of CFUs in Bacterial suspension—No. of CFUs in the gelled disinfectant)/No. of CFUs in Bacterial suspension]×100 and expressed in percentage.

### Example 42. Sample Collection-Disinfection-Disposal Devices for Fluid Samples

An all-in-one sample collection-disinfection-disposal device for fluid samples was prototyped as follows: Three plastic collection vials were mounted one on top of the other such that the top vial contained polyglutamic acid solution (100 mg/mL with 360 mg sodium hydroxide per mL of the glutamic acid solution), the middle one for sample collection and the bottom one prefilled with the requisite amount of the nanomaterial (2 wt % TiO₂, 50 wt % SiO₂, 2 wt % LaPO₄ or 10 wt % boehmite) sol. The design allows the top compartment to be unscrewed and the samples could be collected in the middle compartment. Once collected sample is tested, the remaining sample could be flocculated, gelled or solidified by initially allowing the sample to mix with the nanomaterial sol by breaking the junction between the middle and bottom compartments followed by the addition of polyglutamic acid from the top compartment by breaking the junction between the top and middle compartments. The mixing of the three fluid mixtures allow for complete pathogenic disinfection as evidenced in Example 37.

### Example 43. Sample Collection-Disinfection-Disposal Devices for Solid Samples

An all-in-one sample collection-disinfection-disposal device for solid samples was prototyped as follows: A plastic collection container for solid samples (Eg: cotton waste) was mounted on its top with another smaller plastic vial such that the top vial contained polyglutamic acid solution (100 mg/mL with 360 mg sodium hydroxide per mL of the glutamic acid solution), and the bottom one was half-filled with the requisite amount of the nanomaterial (2 wt % TiO₂, 50 wt % SiO₂, 2 wt % LaPO₄ or 10 wt % boehmite) sol. The design allows the top compartment to be unscrewed and the solid samples could be collected in the bottom compartment. Once ample number of solid samples are collected in the bottom container, it could be flocculated, gelled or solidified by allowing the sample and the nanomaterial sol to mix with polyglutamic acid solution by breaking the junction between the two compartments. The mixing of the solutions and gelation allow for complete pathogenic disinfection as evidenced in Example 37.

### ADVANTAGES OF THE PRESENT INVENTION

mixture with inherent antimicrobial activity.
instantaneous gelation/flocculation upon mixing.
>99.9% microbial disinfection.
reduces risks of spillage and occupational exposure.
allows to dispose the waste as non-regulated medical waste.
applicable to both fluid as well as solid medical waste decontamination.
safer, easier and cost-effective disposal.
premeasured containers can be used to handle any amount of fluidic waste.
possible to incorporate in an all-in-one sample collection-disinfection-disposal device.

1.-16. (canceled)

17. A flocculant based disinfectant composition comprising:
an aqueous solution of a pH regulated poly amino acid; and
an aqueous sol of a nanomaterial, wherein the aqueous solution of the pH regulated poly amino acid is in a range of 10-20% (v/v) of the aqueous sol of the nanomaterial.

18. The flocculant based disinfectant composition as claimed in claim 17, wherein the composition comprises the aqueous solution of the pH regulated poly amino acid at a concentration of 0.1-2000 mg/mL.

19. The flocculant based disinfectant composition as claimed in claim 17, wherein the pH regulated poly amino acid is poly glutamic acid, wherein the pH is regulated by a base.

20. The flocculant based disinfectant composition as claimed in claim 19, wherein the base is sodium hydroxide, and wherein the sodium hydroxide is present at a concentration of 0.1-2000 mg per mL of the poly glutamic acid.

21. The flocculant based disinfectant composition as claimed in claim 17, wherein the nanomaterial is selected from the group consisting of oxides of titanium, aluminium (boehmite), silicon, and phosphates of lanthanide elements.

22. The flocculant based disinfectant composition as claimed in claim 21, wherein the lanthanide is cerium or lanthanum.

23. The flocculant based disinfectant composition as claimed in claim 17, wherein the nanomaterial is in a range of 0.1-70 wt % as a stable sol in water, and wherein the nanomaterial comprises 2 wt % TiO₂, 50 wt % SiO₂, 2 wt % LaPO₄, 10 wt % boehmite, or a combination thereof.

24. The flocculant based disinfectant composition as claimed in claim 17, wherein the composition is characterized to create a non-pourable environment for free-flowing fluid medical wastes.

25. The flocculant based disinfectant composition as claimed in claim 24, wherein the free-flowing fluid medical wastes comprise saliva, urine, blood and their mixtures.

26. The flocculant based disinfectant composition as claimed in claim 17, wherein the composition is characterized to treat solid medical wastes.

27. The flocculant based disinfectant composition as claimed in claim 26, wherein the solid medical wastes comprise cotton, tissue papers, swabs, and needles.

28. A process for preparing a flocculant based disinfectant composition, the process comprising:
mixing a poly amino acid and a base to form a solution of a pH regulated poly amino acid;
preparing an aqueous sol of a nanomaterial; and
adding the aqueous sol of the nanomaterial to the pH regulated poly amino acid solution to obtain the flocculant based disinfectant composition.

29. The process as claimed in claim 28, wherein the poly amino acid is poly glutamic acid, and the base is sodium hydroxide, and wherein the sodium hydroxide is in a range of 0.1-2000 mg/mL of the poly-glutamic acid.

30. The process as claimed in claim 28, wherein the pH regulated poly-glutamic acid is in a range of 1-30% (v/v) of the aqueous sol of the nanomaterial.

31. The process as claimed in claim 28, wherein a sample to be disinfected is added to the flocculant based disinfectant composition, wherein the sample is a liquid sample or a solid sample, wherein the liquid sample is selected from the group consisting of a salt, a metal salt, an aqueous waste, saliva, urine, blood, and a combination thereof, and wherein the solid sample is selected from the group consisting of cotton, a tissue paper, a needle, a swab, and a combination thereof.

32. A device for disinfecting a sample with a flocculant based disinfectant composition, the device comprising:
an upper system;
a middle system;
a bottom system;
a screw cap connected to the upper system; and
a breakable screw-cap connected between the upper system and the bottom system.

33. The device as claimed in claim 32, wherein the upper system is filled with pH regulated poly glutamic acid, wherein the pH is regulated by sodium hydroxide.

34. The device as claimed in claim 32, wherein the middle system is filled with the sample to be disinfected.

35. The device as claimed in claim 32, wherein the bottom system is filled with a 10-20% (wt./v) sol of a nanomaterial in water.

36. The device as claimed in claim 32, wherein the sample is a solid sample or a liquid sample, wherein the liquid sample is selected from the group consisting of a salt solution, a metal salt solution, an aqueous waste, saliva, urine, blood, and a combination thereof, and wherein the solid sample is selected from the group consisting of cotton, a tissue paper, a needle, a swab, and a combination thereof.
